(19) United States
(12) Patent Application Publication
(10) Pub. No.: US 2025/0171855 A1
(43) Pub. Date: May 29, 2025

(54) METHODS FOR DETERMINING CETUXIMAB SENSITIVITY IN CANCER PATIENTS

(71) Applicant: CROWN BIOSCIENCE (SUZHOU) INC., Suzhou, Jiangsu (CN)

(72) Inventors: Binchen MAO, Suzhou, Jiangsu (CN); Sheng GUO, Suzhou, Jiangsu (CN); Henry Qixiang LI, San Diego, CA (US)

(21) Appl. No.: 18/842,791

(22) PCT Filed: Mar. 1, 2023
(86) PCT No.: PCT/CN2023/078908

§ 371 Date: Aug. 29, 2024

(30) Foreign Application Priority Data
Mar. 1, 2022 (WO) PCT/CN2022/078536

(51) Int. Cl. C12Q 1/6886

(52) U.S. Cl. CPC C12Q 1/6886; C12Q 2600/106; C12Q 2600/156; C12Q 2600/158

(57) ABSTRACT

The invention described in the disclosure relates to a panel of biomarkers for determining the responsiveness of a cancer patient treated or to be treated by cetuximab. The disclosure provides methods and compositions, e.g., kits, for evaluating the biomarkers and methods of using such biomarkers to predict a cancer patient's response to cetuximab. Such information can be used in determining prognosis and treatment options for gastric cancer patients.

### CROSS-REFERENCE TO RELATED APPLICATIONS

This application claims priority to application PCT/CN2022/078536, filed Mar. 1, 2022, the disclosure of which is incorporated herein by reference.

### FIELD OF THE INVENTION

The present invention generally relates to cancer diagnostic and treatment.

### BACKGROUND

The epidermal growth factor receptor (EGFR, also named as ErbB-1, and HER1 in human) is a transmembrane receptor protein in the ErbB family of receptors that include four closely related receptor tyrosine kinases: EGFR (ErbB-1), HER2/neu (ErbB-2), Her 3 (ErbB-3) and Her 4 (ErbB-4). The EGFR is activated by binding to its ligands such as epidermal growth factor or transforming growth factor-alpha, resulting in homodimerization or heterodimerization with another member of the EGFR family. This receptor activation is followed by phosphorylation of specific tyrosine residues within the cytoplasmic tail, stimulating the downstream signaling pathway that regulates cell proliferation, migration, adhesion, differentiation and survival.

Gene amplification, mutated activation and/or protein overexpression of EGFR have been observed in a variety of solid tumors, including lung, colorectal, urinary bladder, breast, head, neck, esophageal and gastric carcinomas. In some tumors such as non-small cell lung carcinoma and colorectal carcinoma, increased EGFR expression is associated with advanced stage and an unfavorable prognosis. Many EGFR-targeted drugs, notably tyrosine kinase inhibitors (TKIs), anti-EGFR antibodies and antibody drug conjugates (ADCs), were developed in the past two decades.

Cetuximab (trade name Erbitux™ in the US and Canada), a recombinant human/mouse chimeric monoclonal antibody against EGFR, is the oldest EGFR-targeted monoclonal antibody drug. Cetuximab has been approved for treating EGFR-expressing metastatic colorectal cancer (mCRC) without activating KRAS mutation, and squamous cell carcinoma of the head and neck (SCCHN). However, clinical trials of cetuximab for other cancer type, including non-small cell lung cancer (NSCLC), gastric cancer and esophagus cancer, failed at late stage of clinical trials.

Clinical responses to anticancer therapies are often restricted to a subset of patients. Among the cancer types non-approved for cetuximab treatment, significant responsive population clearly exist according to the non-clinical and clinical trial data. To maximize the efficiency of anticancer therapy using cetuximab, biomarker guided patient stratification has been proposed. However, the identification of predictive biomarkers capable of predicting response to cetuximab still remains a challenge. For example, EGFR as a single gene biomarker could fail under at least two scenarios: the expression level of EGFR is within the medium range or EGFR gene carries a deleterious mutation. Therefore, there is a need to identify new biomarkers to accurately predict the response to cetuximab in cancer patients.

### SUMMARY OF INVENTION

In one aspect, the present disclosure provides a method for predicting cetuximab sensitivity in a patient having cancer. In some embodiments, the method comprising: measuring in a tumor sample from the patient a set of biomarkers selected from EGFR expression level, TMEM40 expression level, ILIA expression level, PTPRN2 expression level, LCE2A expression level, TREM2 expression level, LY6D expression level, TMEM63B expression level, EIF4EBP1 expression level, C20orf56 expression level, SHC expression level, DSG3 expression level, HES6 expression level, FAM25B expression level, PNMA2 expression level, GSK3B expression level, PPM1H expression level, TOX3 expression level, TYMP expression level, Anxa8L2 expression level, ACP6 expression level KRAS mutation, APC mutation, MACF1 mutation, NCOR2 mutation, LPP mutation and a combination thereof; and determining a likelihood of the patient being responsive to cetuximab based on the measured set of biomarkers using a machine learning classifier.

In some embodiments, the cancer is selected from colon cancer, gastric cancer, lung cancer, head and neck cancer and esophagus cancer.

In one aspect, the present disclosure provides a method for predicting cetuximab sensitivity in a patient having colon cancer. In some embodiments, the method comprising: measuring in a tumor sample from the patient a set of biomarkers comprising: EGFR expression level, GSK3B expression level, KRAS mutation, LY6D expression level, PNMA2 expression level, C20orf56 expression level, MACF1 mutation, and NCOR2 mutation; and determining a likelihood of the patient being responsive to cetuximab based on the measured set of biomarkers using a machine learning classifier.

In one aspect, the present disclosure provides a method for predicting cetuximab sensitivity in a patient having gastric cancer. In some embodiments, the method comprising: measuring in a tumor sample from the patient a set of biomarkers comprising: LPP mutation, EHBP1L1 expression level, EGFR expression level, LY6D expression level, C20orf56 expression level, PTPRN2 expression level, FMOD expression level, and NCOR2 mutation; and determining a likelihood of the patient being responsive to cetuximab based on the measured set of biomarkers using a machine learning classifier.

In one aspect, the present disclosure provides a method for predicting cetuximab sensitivity in a patient having lung cancer. In some embodiments, the method comprising: measuring in a tumor sample from the patient a set of biomarkers comprising: LPP mutation, FMOD expression level, EGFR expression level, GSK3B expression level, FAM25B expression level, SHC3 expression level, IL1A expression level, S100A7A expression level, PTPRN2 expression level, and AKT3 expression level; and determining a likelihood of the patient being responsive to cetuximab based on the measured set of biomarkers using a machine learning classifier.

In one aspect, the present disclosure provides a method for predicting cetuximab sensitivity in a patient having head and neck cancer, the method comprising: measuring in a tumor sample from the patient a set of biomarkers comprising: SHC3 expression level, LPP mutation, HES6 expression level, S100A7A expression level, GSK3B expression level, and FAM25B expression level; and determining a likelihood of the patient being responsive to cetuximab based on the measured set of biomarkers using a machine learning classifier.

In one aspect, the present disclosure provides a method for predicting cetuximab sensitivity in a patient having esophagus cancer. In some embodiments, the method comprising: measuring in a tumor sample from the patient a set of biomarkers comprising: LPP mutation, EGFR expression level, FMOD expression level, LY6D expression level, FAM25B expression level, PNMA2 expression level, TOX3 expression level, and PTPRN2 expression level; and determining a likelihood of the patient being responsive to cetuximab based on the measured set of biomarkers using a machine learning classifier.

In some embodiments, the LPP mutation is selected from: R22Q, S123Y, P136S, A174T, and G379E.

In some embodiment, the biomarkers are measured by an amplification assay, a hybridization assay, a sequencing assay or an array.

In some embodiments, the machine learning classifier is built by regularized regression method.

In some embodiments, the method disclosed herein further comprises administering cetuximab to the patient.

In one aspect, the present disclosure provides a non-transitory computer readable medium having instructions stored thereon, wherein the instructions, when executed by a processor, cause the processor to: retrieve data of a set of biomarkers obtained from a tumor sample from a patient having a cancer, wherein the set of biomarkers selected from EGFR expression level, TMEM40 expression level, ILIA expression level, PTPRN2 expression level, LCE2A expression level, TREM2 expression level, LY6D expression level, TMEM63B expression level, EIF4EBP1 expression level, C20orf56 expression level, SHC expression level, DSG3 expression level, HES6 expression level, FAM25B expression level, PNMA2 expression level, GSK3B expression level, PPM1H expression level, TOX3 expression level, TYMP expression level, Anxa8L2 expression level, or ACP6 expression level, KRAS mutation, APC mutation, MACF1 mutation, NCOR2 mutation, LPP mutation and a combination thereof; and determine a likelihood of the patient being responsive to cetuximab based on the data of the set of biomarkers using a machine learning classifier.

In one aspect, the present disclosure provides a non-transitory computer readable medium having instructions stored thereon, wherein the instructions, when executed by a processor, cause the processor to: retrieve data of a set of biomarkers obtained from a tumor sample from a patient having a colon cancer, wherein the set of biomarkers comprises KRAS mutation, LY6D expression level, PNMA2 expression level, C20orf56 expression level, MACF1 mutation, EGFR expression level, GSK3B expression level, and NCOR2 mutation; and determining a likelihood of the patient being responsive to cetuximab based on the data of the set of biomarkers using a machine learning classifier.

In one aspect, the present disclosure provides a non-transitory computer readable medium having instructions stored thereon, wherein the instructions, when executed by a processor, cause the processor to: retrieve data of a set of biomarkers obtained from a tumor sample from a patient having a gastric cancer, wherein the set of biomarkers comprises LPP mutation, EHBP1L1 expression level, EGFR expression level, LY6D expression level, C20orf56 expression level, PTPRN2 expression level, FMOD expression level, and NCOR2 mutation; and determining a likelihood of the patient being responsive to cetuximab based on the data of the set of biomarkers using a machine learning classifier.

In one aspect, the present disclosure provides a non-transitory computer readable medium having instructions stored thereon, wherein the instructions, when executed by a processor, cause the processor to: retrieve data of a set of biomarkers obtained from a tumor sample from a patient having a lung cancer, wherein the set of biomarkers comprises LPP mutation, FMOD expression level, EGFR expression level, GSK3B expression level, FAM25B expression level, SHC3 expression level, IL1A expression level, S10A7A expression level, PTPRN2 expression level, and AKT3 expression level; and determining a likelihood of the patient being responsive to cetuximab based on the data of the set of biomarkers using a machine learning classifier.

In one aspect, the present disclosure provides a non-transitory computer readable medium having instructions stored thereon, wherein the instructions, when executed by a processor, cause the processor to: retrieve data of a set of biomarkers obtained from a tumor sample from a patient having a head and neck cancer, wherein the set of biomarkers comprises SHC3 expression level, LPP mutation, HES6 expression level, S100A7A expression level, GSK3B expression level, and FAM25B expression level; and determining a likelihood of the patient being responsive to cetuximab based on the data of the set of biomarkers using a machine learning classifier.

In one aspect, the present disclosure provides a non-transitory computer readable medium having instructions stored thereon, wherein the instructions, when executed by a processor, cause the processor to: retrieve data of a set of biomarkers obtained from a tumor sample from a patient having a esophagus cancer, wherein the set of biomarkers comprises LPP mutation, EGFR expression level, FMOD expression level, LY6D expression level, FAM25B expression level, PNMA2 expression level, TOX3 expression level, and PTPRN2 expression level; and determining a likelihood of the patient being responsive to cetuximab based on the data of the set of biomarkers using a machine learning classifier.

In one aspect, the present disclosure provides method of generating a machine learning model for predicting sensitivity to an agent in a patient having a cancer, the method comprising steps of: obtaining whole genome expression levels from each of a group of tumor models, wherein the tumor models have been tested for responsiveness to the agent; selecting a first group of genes whose expression levels increase in the tumor models responsive to the agent when compared to the tumor models not responsive to the agent; selecting a second group of genes whose expression levels decrease in the tumor models responsive to the agent when compared to the tumor models not responsive to the agent; selecting a set of biomarkers from the first and the second group of genes using a regularized regression method; and building a machine learning classifier using a logistic regression model. In some embodiments, the agent is cetuximab. In some embodiments, the tumor models are xenograft models.

In some embodiments, the first and the second group of genes are selected by correlation between gene expression level and AUCr or by model performance of ROC metric.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 shows the adding interaction term between EGFR and other genes greatly improves model fit.

FIG. 2 shows that EGFR pathway genes are important in building a model for predicting cetuximab responsiveness in colon cancer patients.

FIG. 3 shows that EGFR pathway genes are important in building a model for predicting cetuximab responsiveness in head and neck cancer patients.

FIG. 4 shows the machine learning model for predicting cetuximab responsiveness in colon cancer.

FIG. 5 shows the machine learning model for predicting cetuximab responsiveness in gastric cancer.

FIG. 6 shows the machine learning model for predicting cetuximab responsiveness in lung cancer.

FIG. 7 shows the machine learning model for predicting cetuximab responsiveness in head and neck cancer.

FIG. 8 shows the machine learning model for predicting cetuximab responsiveness in esophagus cancer.

### DETAILED DESCRIPTION OF THE INVENTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

### Definitions

The following definitions are provided to assist the reader. Unless otherwise defined, all terms of art, notations and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not necessarily be construed to represent a substantial difference over the definition of the term as generally understood in the art.

As used herein, the singular forms “a”, “an” and “the” include plural references unless the context clearly dictates otherwise.

The term “amount” or “level” refers to the quantity of a polynucleotide of interest or a polypeptide of interest present in a sample. Such quantity may be expressed in the absolute terms, i.e., the total quantity of the polynucleotide or polypeptide in the sample, or in the relative terms, i.e., the concentration of the polynucleotide or polypeptide in the sample.

As used herein, the term “cancer” refers to any diseases involving an abnormal cell growth and include all stages and all forms of the disease that affects any tissue, organ or cell in the body. The term includes all known cancers and neoplastic conditions, whether characterized as malignant, benign, soft tissue, or solid, and cancers of all stages and grades including pre- and post-metastatic cancers. In general, cancers can be categorized according to the tissue or organ from which the cancer is located or originated and morphology of cancerous tissues and cells. As used herein, cancer types include, without limitation, acute lymphoblastic leukemia (ALL), acute myeloid leukemia, adrenocortical carcinoma, anal cancer, astrocytoma, childhood cerebellar or cerebral, basal-cell carcinoma, bile duct cancer, bladder cancer, bone tumor, brain cancer, cerebellar astrocytoma, cerebral astrocytoma/malignant glioma, ependymoma, medulloblastoma, supratentorial primitive neuroectodermal tumors, visual pathway and hypothalamic glioma, breast cancer, Burkitt's lymphoma, cervical cancer, chronic lymphocytic leukemia, chronic myelogenous leukemia, colon cancer, emphysema, endometrial cancer, ependymoma, esophageal cancer, Ewing's sarcoma, retinoblastoma, gastric (stomach) cancer, glioma, head and neck cancer, heart cancer, Hodgkin lymphoma, islet cell carcinoma (endocrine pancreas), Kaposi sarcoma, kidney cancer (renal cell cancer), laryngeal cancer, leukaemia, liver cancer, lung cancer, neuroblastoma, non-Hodgkin lymphoma, ovarian cancer, pancreatic cancer, pharyngeal cancer, prostate cancer, rectal cancer, renal cell carcinoma (kidney cancer), retinoblastoma, Ewing family of tumors, skin cancer, stomach cancer, testicular cancer, throat cancer, thyroid cancer, vaginal cancer.

It is noted that in this disclosure, terms such as “comprises”, “comprised”, “comprising”, “contains”, “containing” and the like have the meaning attributed in United States Patent law; they are inclusive or open-ended and do not exclude additional, un-recited elements or method steps. Terms such as “consisting essentially of” and “consists essentially of” have the meaning attributed in United States Patent law; they allow for the inclusion of additional ingredients or steps that do not materially affect the basic and novel characteristics of the claimed invention. The terms “consists of” and “consisting of” have the meaning ascribed to them in United States Patent law; namely that these terms are close ended.

A “cell”, as used herein, can be prokaryotic or eukaryotic. A prokaryotic cell includes, for example, bacteria. A eukaryotic cell includes, for example, a fungus, a plant cell, and an animal cell. The types of an animal cell (e.g., a mammalian cell or a human cell) includes, for example, a cell from circulatory/immune system or organ (e.g., a B cell, a T cell (cytotoxic T cell, natural killer T cell, regulatory T cell, T helper cell), a natural killer cell, a granulocyte (e.g., basophil granulocyte, an eosinophil granulocyte, a neutrophil granulocyte and a hypersegmented neutrophil), a monocyte or macrophage, a red blood cell (e.g., reticulocyte), a mast cell, a thrombocyte or megakaryocyte, and a dendritic cell); a cell from an endocrine system or organ (e.g., a thyroid cell (e.g., thyroid epithelial cell, parafollicular cell), a parathyroid cell (e.g., parathyroid chief cell, oxyphil cell), an adrenal cell (e.g., chromaffin cell), and a pineal cell (e.g., pinealocyte)); a cell from a nervous system or organ (e.g., a glioblast (e.g., astrocyte and oligodendrocyte), a microglia, a magnocellular neurosecretory cell, a stellate cell, a boettcher cell, and a pituitary cell (e.g., gonadotrope, corticotrope, thyrotrope, somatotrope, and lactotroph)); a cell from a respiratory system or organ (e.g., a pneumocyte (a type I pneumocyte and a type II pneumocyte), a clara cell, a goblet cell, an alveolar macrophage); a cell from circular system or organ (e.g., myocardiocyte and pericyte); a cell from digestive system or organ (e.g., a gastric chief cell, a parietal cell, a goblet cell, a paneth cell, a G cell, a D cell, an ECL cell, an I cell, a K cell, an S cell, an enteroendocrine cell, an enterochromaffin cell, an APUD cell, a liver cell (e.g., a hepatocyte and Kupffer cell)); a cell from integumentary system or organ (e.g., a bone cell (e.g., an osteoblast, an osteocyte, and an osteoclast), a teeth cell (e.g., a cementoblast, and an ameloblast), a cartilage cell (e.g., a chondroblast and a chondrocyte), a skin/hair cell (e.g., a trichocyte, a keratinocyte, and a melanocyte (Nevus cell)), a muscle cell (e.g., myocyte), an adipocyte, a fibroblast, and a tendon cell), a cell from urinary system or organ (e.g., a podocyte, a juxtaglomerular cell, an intraglomerular mesangial cell, an extraglomerular mesangial cell, a kidney proximal tubule brush border cell, and a macula densa cell), and a cell from reproductive system or organ (e.g., a spermatozoon, a Sertoli cell, a leydig cell, an ovum, an oocyte). A cell can be normal, healthy cell; or a diseased or unhealthy cell (e.g., a cancer cell). A cell further includes a mammalian zygote or a stem cell which include an embryonic stem cell, a fetal stem cell, an induced pluripotent stem cell, and an adult stem cell. A stem cell is a cell that is capable of undergoing cycles of cell division while maintaining an undifferentiated state and differentiating into specialized cell types. A stem cell can be an omnipotent stem cell, a pluripotent stem cell, a multipotent stem cell, an oligopotent stem cell and a unipotent stem cell, any of which may be induced from a somatic cell. A stem cell may also include a cancer stem cell. A mammalian cell can be a rodent cell, e.g., a mouse, rat, hamster cell. A mammalian cell can be a lagomorpha cell, e.g., a rabbit cell. A mammalian cell can also be a primate cell, e.g., a human cell. In certain examples, the cells are those used for mass bioproduction, e.g., CHO cells.

The terms “determining,” “assessing,” “assaying,” “measuring” and “detecting” can be used interchangeably and refer to both quantitative and semi-quantitative determinations. Where either a quantitative and semi-quantitative determination is intended, the phrase “determining a level” of a polynucleotide or polypeptide of interest or “detecting” a polynucleotide or polypeptide of interest can be used.

The term “gene product” or “gene expression product” refers to an RNA or protein encoded by the gene.

The term “hybridizing” refers to the binding, duplexing, or hybridizing of a nucleic acid molecule preferentially to a particular nucleotide sequence under stringent conditions. The term “stringent conditions” refers to conditions under which a probe will hybridize preferentially to its target subsequence, and to a lesser extent to, or not at all to, other sequences in a mixed population (e.g., a cell lysate or DNA preparation from a tissue biopsy). A “stringent hybridization” and “stringent hybridization wash conditions” in the context of nucleic acid hybridization (e.g., as in array, microarray, Southern or northern hybridizations) are sequence dependent, and are different under different environmental parameters. An extensive guide to the hybridization of nucleic acids is found in, e.g., Tijssen Laboratory Techniques in Biochemistry and Molecular Biology—Hybridization with Nucleic Acid Probes part I, Ch. 2, “Overview of principles of hybridization and the strategy of nucleic acid probe assays,” (1993) Elsevier, N.Y. Generally, highly stringent hybridization and wash conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Very stringent conditions are selected to be equal to the Tm for a particular probe. An example of stringent hybridization conditions for hybridization of complementary nucleic acids which have more than 100 complementary residues on an array or on a filter in a Southern or northern blot is 42° C. using standard hybridization solutions (see, e.g., Sambrook and Russell Molecular Cloning: A Laboratory Manual (3rd ed.) Vol. 1-3 (2001) Cold Spring Harbor Laboratory, Cold Spring Harbor Press, NY). An example of highly stringent wash conditions is 0.15 M NaCl at 72° C. for about 15 minutes. An example of stringent wash conditions is a 0.2×SSC wash at 65° C. for 15 minutes. Often, a high stringency wash is preceded by a low stringency wash to remove background probe signal. An example medium stringency wash for a duplex of, e.g., more than 100 nucleotides, is 1×SSC at 45° C. for 15 minutes. An example of a low stringency wash for a duplex of, e.g., more than 100 nucleotides, is 4×SSC to 6×SSC at 40° C. for 15 minutes.

The term “nucleic acid” and “polynucleotide” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, shRNA, single-stranded short or long RNAs, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

The term “overall survival” refers to the time interval from either the time of diagnosis or the start of treatment that the patient is still alive.

The term “prognose” or “prognosing” as used herein refers to the prediction or forecast of the future course or outcome of a disease or condition.

The term “progression-free survival” refers to the time interval from treatment of the patient until the progression of cancer or death of the patient, whichever occurs first.

In general, a “protein” is a polypeptide (i.e., a string of at least two amino acids linked to one another by peptide bonds). Proteins may include moieties other than amino acids (e.g., may be glycoproteins) and/or may be otherwise processed or modified. Those of ordinary skill in the art will appreciate that a “protein” can be a complete polypeptide chain as produced by a cell (with or without a signal sequence), or can be a functional portion thereof. Those of ordinary skill will further appreciate that a protein can sometimes include more than one polypeptide chain, for example linked by one or more disulfide bonds or associated by other means.

The term “recommending” or “suggesting” in the context of a treatment of a disease, refers to making a suggestion or a recommendation for therapeutic intervention (e.g., drug therapy, adjunctive therapy, etc.) and/or disease management which are specifically applicable to the patient.

The terms “responsive,” “clinical response,” “positive clinical response,” and the like, as used in the context of a patient's response to a cancer therapy, are used interchangeably and refer to a favorable patient response to a treatment as opposed to unfavorable responses, i.e., adverse events. In a patient, beneficial response can be expressed in terms of a number of clinical parameters, including loss of detectable tumor (complete response, CR), decrease in tumor size and/or cancer cell number (partial response, PR), tumor growth arrest (stable disease, SD), enhancement of anti-tumor immune response, possibly resulting in regression or rejection of the tumor; relief, to some extent, of one or more symptoms associated with the tumor; increase in the length of survival following treatment; and/or decreased mortality at a given point of time following treatment. Continued increase in tumor size and/or cancer cell number and/or tumor metastasis is indicative of lack of beneficial response to treatment. In a population the clinical benefit of a drug, i.e., its efficacy can be evaluated on the basis of one or more endpoints. For example, analysis of overall response rate (ORR) classifies as responders those patients who experience CR or PR after treatment with drug. Analysis of disease control (DC) classifies as responders those patients who experience CR, PR or SD after treatment with drug. A positive clinical response can be assessed using any endpoint indicating a benefit to the patient, including, without limitation, (1) inhibition, to some extent, of tumor growth, including slowing down and complete growth arrest; (2) reduction in the number of tumor cells; (3) reduction in tumor size; (4) inhibition (i.e., reduction, slowing down or complete stopping) of tumor cell infiltration into adjacent peripheral organs and/or tissues; (5) inhibition of metastasis; (6) enhancement of anti-tumor immune response, possibly resulting in regression or rejection of the tumor; (7) relief, to some extent, of one or more symptoms associated with the tumor; (8) increase in the length of survival following treatment; and/or (9) decreased mortality at a given point of time following treatment. Positive clinical response may also be expressed in terms of various measures of clinical outcome. Positive clinical outcome can also be considered in the context of an individual's outcome relative to an outcome of a population of patients having a comparable clinical diagnosis, and can be assessed using various endpoints such as an increase in the duration of recurrence-free interval (RFI), an increase in the time of survival as compared to overall survival (OS) in a population, an increase in the time of disease-free survival (DFS), an increase in the duration of distant recurrence-free interval (DRFI), and the like. Additional endpoints include a likelihood of any event (AE)-free survival, a likelihood of metastatic relapse (MR)-free survival (MRFS), a likelihood of disease-free survival (DFS), a likelihood of relapse-free survival (RFS), a likelihood of first progression (FP), and a likelihood of distant metastasis-free survival (DMFS). An increase in the likelihood of positive clinical response corresponds to a decrease in the likelihood of cancer recurrence or relapse.

As used herein, the term “subject” refers to a human or any non-human animal (e.g., mouse, rat, rabbit, dog, cat, cattle, swine, sheep, horse or primate). A human includes pre and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

The term “tumor sample” includes a biological sample or a sample from a biological source that contains one or more tumor cells. Biological samples include samples from body fluids, e.g., blood, plasma, serum, or urine, or samples derived, e.g., by biopsy, from cells, tissues or organs, preferably tumor tissue suspected to include or essentially consist of cancer cells.

The term “treatment,” “treat,” or “treating” refer to a method of reducing the effects of a cancer (e.g., breast cancer, lung cancer, ovarian cancer or the like) or symptom of cancer. Thus, in the disclosed method, treatment can refer to a 10%, 20%, 30%, 40%, 50%, 60%, 70%), 80%), 90%), or 100% reduction in the severity of an cancer or symptom of the cancer. For example, a method of treating a disease is considered to be a treatment if there is a 10% reduction in one or more symptoms of the disease in a subject as compared to a control. Thus, the reduction can be a 10%, 20%, 30%, 40%, 50%, 60%, 70%, 80%, 90%, 100% or any percent reduction between 10 and 100% as compared to native or control levels. It is understood that treatment does not necessarily refer to a cure or complete ablation of the disease, condition, or symptoms of the disease or condition.

### Biomarkers for Predicting Response to Cetuximab

The methods and compositions described herein are based, in part, on the discovery of a panel of biomarkers correlated with cetuximab sensitivity in a cancer patient. In one aspect, the present disclosure provides methods for predicting response to cetuximab in a patient having gastric cancer. In certain embodiments, the biomarkers include expression level of certain genes and certain gene mutations. In certain embodiments, the biomarkers are selected from: EGFR expression level, TMEM40 expression level, ILIA expression level, PTPRN2 expression level, LCE2A expression level, TREM2 expression level, LY6D expression level, TMEM63B expression level, EIF4EBP1 expression level, KRAS mutation, APC mutation, MACF1 mutation, NCOR2 mutation, LPP mutation, C20orf56 expression level, SHC expression level, DSG3 expression level, HES6 expression level, FAM25B expression level, PNMA2 expression level, GSK31B expression level, PPM1H expression level, TOX3 expression level, TYMP expression level, Anxa8L2 expression level, and ACP6 expression level. The information of certain biomarkers can be found in Table 1. The mRNA and protein sequences are referred to NCBI RefSeq No.

TABLE 1
Information of Biomarkers

| Gene Name | mRNA Sequence | Protein Sequence | Mutation |
|---|---|---|---|
| EGFR | NM_001346941.1 | NP_005219.2 | |
| | NM_005228.4 | NP_958439.1 | |
| | NM_001346898.1 | NP_958440.1 | |
| | NM_001346900.1 | NP_958441.1 | |
| | NM_001346899.1 | NP_001333826.1 | |
| | NM_001346897.1 | NP_001333828.1 | |
| | NM_201284.1 | NP_001333829.1 | |
| | NM_201283.1 | NP_001333827.1 | |
| | NM_201282.1 | NP_001333870.1 | |
| TMEM40 | NM_001284406. | NP_001271335.1 | |
| | NM_001284407.1 | NP_001271336.1 | |
| | NM_001284408.1 | NP_001271337.1 | |
| | NM_018306.3 | NP_060776.2 | |
| | XM_011533937.2 | XP_011532239.1 | |
| IL1A | NM_000575.4 | NP_000566.3 | |
| PTPRN2 | NM_001308267.1 | NP_001295196.1 | |
| | NM_001308268.1 | NP_001295197.1 | |
| | NM_002847.4 | NP_002838.2 | |
| | NM_130842.3 | NP_570857.2 | |
| | NM_130843.3 | NP_570858.2 | |
| LCE2A | NM_178428.3 | NP_848515.1 | |
| TREM2 | NM_001271821.1 | NP_001258750.1 | |
| | NM_018965.3 | NP_061838.1 | |
| LY6D | NM_003695.2 | NP_003686.1 | |
| TMEM63B | NM_001318792.1 | NP_001305721.1 | |
| | NM_018426.2 | NP_060896.1 | |
| | XM_005249213.4 | XP_005249270.1 | |
| | XM_005249217.1 | XP_005249274.1 | |
| | XM_006715135.1 | XP_006715198.1 | |
| | XM_017011000.1 | XP_016866489.1 | |
| EIF4EBP1 | NM_004095.3 | NP_004086.1 | |
| C20orf56 | NR_001558* | | |
| SHC3 | NM_016848.5 | NP_058544.3 | |
| DSG3 | NM_001944.2 | NP_001935.2 | |
| HES6 | NM_001142853.2 | NP_001136325.1 | |
| | NM_001282434.1 | NP_001269363.1 | |
| | NM_018645.5 | NP_061115.2 | |
| FAM25B | NR_104039* | | |
| PNMA2 | NM_007257.5 | NP_009188.1 | |
| | XM_011544365.2 | XP_011542667.1 | |
| GSK3B | NM_001146156.1 | NP_001139628.1 | |
| | NM_002093.3 | NP_002084.2 | |
| PPM1H | NM_020700.1 | NP_065751.1 | |
| TOX3 | NM_001080430.3 | NP_001073899.2 | |
| | NM_001146188.2 | NP_001139660.1 | |
| TYMP | NM_001113755.2 | NP_001107227.1 | |
| | NM_001113756.2 | NP_001107228.1 | |
| | NM_001257988.1 | NP_001244917.1 | |
| | NM_001257989.1 | NP_001244918.1 | |
| | NM_001953.4 | NP_001944.1 | |
| Anxa8L2 | NM_001098845.2 | NP_001092315.2 | |
| | NM_001278924.1 | NP_001265853.1 | |
| ACP6 | NM_016361.4 | NP_057445.4 | |
| EHBP1L1 | NM_053252.3 | NP_444482.2 | |
| FMOD | NM_002023.4 | NP_002014.2 | |
| S100A7A | NM_176823.3 | NP_789793.1 | |
| AKT3 | NM_001206729.1 | NP_001193658.1 | |
| | NM_005465.4 | NP_005456.1 | |
| | NM_181690.2 | NP_859029.1 | |
| | XM_005272994.4 | XP_005273051.1 | |
| | XM_005272995.2 | XP_005273052.1 | |
| KRAS | NM_004985.4 | NP_004976.2 | K5E, K5N, G10GG, G12A, G12C, |
| | NM_033360.3 | NP_203524.1 | G12D, G12R, G12S, G12V, G13D, |
| | XM_006719069.3 | XP_006719132.1 | G13R, V14I, L19F, Q22E, P34L, |
| | XM_011520653.2 | XP_011518955.1 | P34Q, P34R, I36M, T58I, A59T, |
| | | | G60S, Q61H, Y71H, K117N, A146T, |
| | | | A146V, K147E, V152G, D153V, |
| | | | F156I, F156L |
| APC | NM_000038.5 | NP_000029.2 | R99W, S171I, R414C, S722G, S784T, |
| | NM_001127510.2 | NP_001120982.1 | G817C, P870S, I880T, V890I, S906Y, |
| | NM_001127511.2 | NP_001120983.2 | E911G, N942D, Y1027C, E1057G, |
| | | | N1118D, G1120E, R1171C, R1171H, |
| | | | P1176L, A1184P, F1197S, I1254F, |
| | | | I1259T, T1292M, A1296V, I1304V, |
| | | | I1307K, G1312E, T1313A, E1317Q, |
| | | | V1326A, R1348W, S1395C, D1422H, |
| | | | V1472I, S1495G, T1496S, A1508V, |
| | | | V1822D, R1882T, S1973T, V2499L, |
| | | | G2502S, S2621C, I2738T, L2839F |
| MACF1 | NM_012090.5 | NP_036222.3 | E302V, M4357V, K6201R, A6308T, |
| | | | E6462Q, S6628T, G6664R, T6752I, |
| | | | I6855V, G7093E, C7135F, D7186Y, |
| | | | C7188F, C7188G |
| NCOR2 | NM_001077261.3 | NP_001070729.2 | G781E, A1699T, P2001S |
| | NM_001206654.1 | NP_001193583.1 | |
| | NM_006312.5 | NP_006303.4 | |
| LPP | NM_001167671.2 | NP_001161143.1 | R22Q, S123Y, P136S, T146A, A174T, |
| | NM_005578.4 | NP_005569.1 | S259P, Y346H, G379E |
| | XM_005247446.4 | XP_005247503.1 | |
| | XM_005247450.4 | XP_005247507.1 | |
| | XM_005247451.4 | XP_005247508.1 | |
| | XM_005247453.2 | XP_005247510.1 | |
| | XM_011512820.2 | XP_011511122.1 | |
| | XM_011512827.2 | XP_011511129.1 | |
| | XM_011512828.2 | XP_011511130.1 | |
| | XM_011512831.2 | XP_011511133.1 | |
| | XM_011512833.2 | XP_011511135.1 | |
| | XM_011512834.2 | XP_011511136.1 | |
| | XM_017006377.1 | XP_016861866.1 | |
| | XM_017006378.1 | XP_016861867.1 | |
| | XM_017006379.1 | XP_016861868.1 | |
| | XM_017006380.1 | XP_016861869.1 | |

*Long Non-coding RNA

### Measuring Biomarkers

The methods of the present disclosure involve detecting or measuring at least a subset of the predicting biomarkers disclosed herein, in a tumor sample obtained from a patient suspected of having cancer or at risk of having cancer. In some embodiments, the patient has been diagnosed with cancer.

### Sample Preparation

The tumor sample can be a biological sample comprising cancer cells. In some embodiments, the tumor sample is a fresh or archived sample obtained from a tumor, e.g., by a tumor biopsy or fine needle aspirate. The sample also can be any biological fluid containing cancer cells. The tumor sample can be isolated or obtained from any number of primary tumors, including, but not limited to, tumors of the breast, lung, prostate, brain, liver, kidney, intestines, colon, spleen, pancreas, thymus, testis, ovary, uterus, and the like. In some embodiments, the tumor sample is from a tumor cell line. The collection of a tumor sample from a subject is performed in accordance with the standard protocol generally followed by hospital or clinics, such as during a biopsy.

In certain embodiments, the method further comprises isolating or extracting cancer cell (such as circulating tumor cell) from the biological fluid sample (such as peripheral blood sample) or the tissue sample obtained from the subject. The cancer cells can be separated by immunomagnetic separation technology such as that available from Immunicon (Huntingdon Valley, Pa.).

In certain embodiments, a tissue sample can be processed to perform in situ hybridization. For example, the tissue sample can be paraffin-embedded before fixing on a glass microscope slide, and then deparaffinized with a solvent, typically xylene.

In certain embodiments, the method further comprises isolating the nucleic acid, e.g., DNA or RNA from the sample. Various methods of extraction are suitable for isolating the DNA or RNA from cells or tissues, such as phenol and chloroform extraction, and various other methods as described in, for example, Ausubel et al., Current Protocols of Molecular Biology (1997) John Wiley & Sons, and Sambrook and Russell, Molecular Cloning: A Laboratory Manual 3^{rd} ed. (2001).

Commercially available kits can also be used to isolate DNA and/or RNA, including for example, the NucliSens extraction kit (Biomerieux, Marcy l'Etoile, France), QIAamp™ mini blood kit, Agencourt Genfind™, Rneasy® mini columns (Qiagen), PureLink® RNA mini kit (Thermo Fisher Scientific), and Eppendorf Phase Lock Gels™. A skilled person can readily extract or isolate RNA or DNA following the manufacturer's protocol.

### Methods of Measuring Biomarkers

The biomarkers disclosed herein can be detected in the level of DNA (e.g. genomic DNA) or RNA (e.g. mRNA) using proper methods known in the art including, without limitation, amplification assay, hybridization assay, and sequencing assay. The gene expression level can be detected in the RNA (e.g., mRNA) level or protein level using proper methods known in the art. The gene mutations can be detected in the DNA level or RNA level using proper methods known in the art.

### Sequencing Methods

Sequencing methods useful in the measurement of the biomarkers involves sequencing of the target nucleic acid. Any sequencing known in the art can be used to detect the biomarkers of interest. In general, sequencing methods can be categorized to traditional or classical methods and high throughput sequencing (next generation sequencing). Traditional sequencing methods include Maxam-Gilbert sequencing (also known as chemical sequencing) and Sanger sequencing (also known as chain-termination methods).

High throughput sequencing, or next generation sequencing, by using methods distinguished from traditional methods, such as Sanger sequencing, is highly scalable and able to sequence the entire genome or transcriptome at once. High throughput sequencing involves sequencing-by-synthesis, sequencing-by-ligation, and ultra-deep sequencing (such as described in Marguiles et al., Nature 437 (7057): 376-80 (2005)). Sequence-by-synthesis involves synthesizing a complementary strand of the target nucleic acid by incorporating labeled nucleotide or nucleotide analog in a polymerase amplification. Immediately after or upon successful incorporation of a label nucleotide, a signal of the label is measured and the identity of the nucleotide is recorded. The detectable label on the incorporated nucleotide is removed before the incorporation, detection and identification steps are repeated. Examples of sequence-by-synthesis methods are known in the art, and are described for example in U.S. Pat. Nos. 7,056,676, 8,802,368 and 7,169,560, the contents of which are incorporated herein by reference. Sequencing-by-synthesis may be performed on a solid surface (or a microarray or a chip) using fold-back PCR and anchored primers. Target nucleic acid fragments can be attached to the solid surface by hybridizing to the anchored primers, and bridge amplified. This technology is used, for example, in the Illumina® sequencing platform.

Pyrosequencing involves hybridizing the target nucleic acid regions to a primer and extending the new strand by sequentially incorporating deoxynucleotide triphosphates corresponding to the bases A, C, G, and T (U) in the presence of a polymerase. Each base incorporation is accompanied by release of pyrophosphate, converted to ATP by sulfurylase, which drives synthesis of oxyluciferin and the release of visible light. Since pyrophosphate release is equimolar with the number of incorporated bases, the light given off is proportional to the number of nucleotides adding in any one step. The process is repeated until the entire sequence is determined.

In certain embodiments, the biomarkers described herein are detected by whole transcriptome shotgun sequencing (RNA sequencing). The method of RNA sequencing has been described (see Wang Z, Gerstein M and Snyder M, Nature Review Genetics (2009) 10:57-63; Maher C A et al., Nature (2009) 458:97-101; Kukurba K & Montgomery S B, Cold Spring Harbor Protocols (2015) 2015(11): 951-969).

### Amplification Assay

A nucleic acid amplification assay involves copying a target nucleic acid (e.g. DNA or RNA), thereby increasing the number of copies of the amplified nucleic acid sequence. Amplification may be exponential or linear. Exemplary nucleic acid amplification methods include, but are not limited to, amplification using the polymerase chain reaction (“PCR”, see U.S. Pat. Nos. 4,683,195 and 4,683,202; PCR Protocols: A Guide To Methods And Applications (Innis et al., eds, 1990)), reverse transcriptase polymerase chain reaction (RT-PCR), quantitative real-time PCR (qRT-PCR); quantitative PCR, such as TaqMan®, nested PCR, ligase chain reaction (See Abravaya, K., et al., Nucleic Acids Research, 23:675-682, (1995), branched DNA signal amplification (see, Urdea, M. S., et al., AIDS, 7 (suppl 2):S11-S14, (1993), amplifiable RNA reporters, Q-beta replication (see Lizardi et al., Biotechnology (1988) 6: 1197), transcription-based amplification (see, Kwoh et al., Proc. Natl. Acad. Sci. USA (1989) 86: 1173-1177), boomerang DNA amplification, strand displacement activation, cycling probe technology, self-sustained sequence replication (Guatelli et al., Proc. Natl. Acad. Sci. USA (1990) 87:1874-1878), rolling circle replication (U.S. Pat. No. 5,854,033), isothermal nucleic acid sequence based amplification (NASBA), and serial analysis of gene expression (SAGE).

In certain embodiments, the nucleic acid amplification assay is a PCR-based method. PCR is initiated with a pair of primers that hybridize to the target nucleic acid sequence to be amplified, followed by elongation of the primer by polymerase which synthesizes the new strand using the target nucleic acid sequence as a template and dNTPs as building blocks. Then the new strand and the target strand are denatured to allow primers to bind for the next cycle of extension and synthesis. After multiple amplification cycles, the total number of copies of the target nucleic acid sequence can increase exponentially.

In certain embodiments, intercalating agents that produce a signal when intercalated in double stranded DNA may be used. Exemplary agents include SYBR GREEN™ and SYBR GOLD™. Since these agents are not template-specific, it is assumed that the signal is generated based on template-specific amplification. This can be confirmed by monitoring signal as a function of temperature because melting point of template sequences will generally be much higher than, for example, primer-dimers, etc.

In certain embodiments, a detectably labeled primer or a detectably labeled probe can be used, to allow detection of the biomarkers corresponding to that primer or probe. In certain embodiments, multiple labeled primers or labeled probes with different detectable labels can be used to allow simultaneous detection of multiple biomarkers.

### Hybridization Assay

Nucleic acid hybridization assays use probes to hybridize to the target nucleic acid, thereby allowing detection of the target nucleic acid. Non-limiting examples of hybridization assay include Northern blotting, Southern blotting, in situ hybridization, microarray analysis, and multiplexed hybridization-based assays.

In certain embodiments, the probes for hybridization assay are detectably labeled. In certain embodiments, the nucleic acid-based probes for hybridization assay are unlabeled. Such unlabeled probes can be immobilized on a solid support such as a microarray, and can hybridize to the target nucleic acid molecules which are detectably labeled.

In certain embodiments, hybridization assays can be performed by isolating the nucleic acids (e.g., RNA or DNA), separating the nucleic acids (e.g., by gel electrophoresis) followed by transfer of the separated nucleic acid on suitable membrane filters (e.g., nitrocellulose filters), where the probes hybridize to the target nucleic acids and allows detection. See, for example, Molecular Cloning: A Laboratory Manual, J. Sambrook et al., eds., 2nd edition, Cold Spring Harbor Laboratory Press, 1989, Chapter 7. The hybridization of the probe and the target nucleic acid can be detected or measured by methods known in the art. For example, autoradiographic detection of hybridization can be performed by exposing hybridized filters to photographic film.

In some embodiments, hybridization assays can be performed on microarrays. Microarrays provide a method for the simultaneous measurement of the levels of large numbers of target nucleic acid molecules. The target nucleic acids can be RNA, DNA, cDNA reverse transcribed from mRNA, or chromosomal DNA. The target nucleic acids can be allowed to hybridize to a microarray comprising a substrate having multiple immobilized nucleic acid probes arrayed at a density of up to several million probes per square centimeter of the substrate surface. The RNA or DNA in the sample is hybridized to complementary probes on the array and then detected by laser scanning. Hybridization intensities for each probe on the array are determined and converted to a quantitative value representing relative levels of the RNA or DNA. See, U.S. Pat. Nos. 6,040,138, 5,800,992 and 6,020,135, 6,033,860, and 6,344,316.

Techniques for the synthesis of these arrays using mechanical synthesis methods are described in, e.g., U.S. Pat. No. 5,384,261. Although a planar array surface is often employed the array may be fabricated on a surface of virtually any shape or even a multiplicity of surfaces. Arrays may be peptides or nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992. Arrays may be packaged in such a manner as to allow for diagnostics or other manipulation of an all-inclusive device. Useful microarrays are also commercially available, for example, microarrays from Affymetrix, from Nano String Technologies, QuantiGene 2.0 Multiplex Assay from Panomics.

In certain embodiments, hybridization assays can be in situ hybridization assay. In situ hybridization assay is useful to detect the presence of gene mutations. Probes useful for in situ hybridization assay can be mutation specific probes, which hybridize to a specific gene mutation to detect the presence or absence of the specific mutation of interest. Methods for use of unique sequence probes for in situ hybridization are described in U.S. Pat. No. 5,447,841, incorporated herein by reference. Probes can be viewed with a fluorescence microscope and an appropriate filter for each fluorophore, or by using dual or triple band-pass filter sets to observe multiple fluorophores. See, e.g., U.S. Pat. No. 5,776,688 to Bittner, et al., which is incorporated herein by reference. Any suitable microscopic imaging method can be used to visualize the hybridized probes, including automated digital imaging systems. Alternatively, techniques such as flow cytometry can be used to examine the hybridization pattern of the probes.

### Immunoassay

Immunoassays used herein typically involves using antibodies that specifically bind to biomarker protein. Such antibodies can be obtained using methods known in the art (see, e.g., Huse et al., Science (1989) 246:1275-1281; Ward et al, Nature (1989) 341:544-546), or can be obtained from commercial sources. Examples of immunoassays include, without limitation, Western blotting, enzyme-linked immunosorbent assay (ELISA), enzyme immunoassay (EIA), radioimmunoassay (RIA), immunoprecipitations, sandwich assays, competitive assays, immunofluorescent staining and imaging, immunohistochemistry (IHC), and fluorescent activating cell sorting (FACS). For a review of immunological and immunoassay procedures, see Basic and Clinical Immunology (Stites & Terr eds., 7^{th} ed. 1991). Moreover, the immunoassays can be performed in any of several configurations, which are reviewed extensively in Enzyme Immunoassay (Maggio, ed., 1980); and Harlow & Lane, supra. For a review of the general immunoassays, see also Methods in Cell Biology: Antibodies in Cell Biology, volume 37 (Asai, ed. 1993); Basic and Clinical Immunology (Stites & Terr, eds., 7^{th} ed. 1991).

Any of the assays and methods provided herein for the measurement of the gene expression level can be adapted or optimized for use in automated and semi-automated systems, or point of care assay systems.

The gene expression level described herein can be normalized using a proper method known in the art. For example, the gene expression level can be normalized to a standard level of a standard marker, which can be predetermined, determined concurrently, or determined after a sample is obtained from the subject. The standard marker can be run in the same assay or can be a known standard marker from a previous assay. For another example, the gene expression level can be normalized to an internal control which can be an internal marker, or an average level or a total level of a plurality of internal markers.

The level of mRNA expression of each of the biomarkers described herein can be normalized to a reference level for a control gene. The control value can be predetermined, determined concurrently, or determined after a sample is obtained from the subject. The standard can be run in the same assay or can be a known standard from a previous assay. In the cases when the level of RNA expression is determined by RNA sequencing, the level of RNA expression of each of the biomarkers can be normalized to the total reads of the sequencing. The normalized levels of mRNA expression of the biomarker genes can be transformed in to a score, e.g., using the methods and models described herein.

### Methods for Predicting Response to Cetuximab

After measuring the panel of biomarkers, the method disclosed herein includes determining a likelihood of the patient being responsive to cetuximab. In certain embodiments, the likelihood can be determined based on models using machine learning techniques such as partial least square (Wold S et al., PLS for Multivariate Linear Modeling, Chemometric Methods in Molecular Design (1995) Han van de Waterbeemd (ed.), pp. 195-218. VCH, Weinheim), elastic net (Zou H et al., Regularization and Variable Selection via the Elastic Net, Journal of the Royal Statistical Society, Series B (2005) 67(2): 301-320), support vector machine (Vapnik V, The Nature of Statistical Learning Theory (2010) Springer), random forest (Breiman L, Random Forests, Machine Learning (2001) 45: 5-32), neural net (Bishop C, Neural Networks for Pattern Recognition (1995) Oxford University Press, Oxford) and gradient boosting machine (Friedman J, Greedy Function Approximation: A Gradient Boosting Machine, Annals of Statistics (2001) 29(5), 1189-1232). In one case, the likelihood is determined based on models built by regularized regression method.

As used herein, “machine learning” refers to a computer-implemented technique that gives computer systems the ability to progressively improve performance on a specific task with data, i.e., to learn from the data, without being explicitly programmed. Machine learning technique adopts algorithms that can learn from and make prediction on data through building a model, i.e., a description of a system using mathematical concepts, from sample inputs. A core objective of machine learning is to generalize from the experience, i.e., to perform accurately on new data after having experienced a learning data set. In the context of biomedical diagnosis or prognosis, machine learning techniques generally involves supervised learning process, in which the computer is presented with example inputs (e.g., signature of gene expression) and their desired outputs (e.g., responsiveness) to learn a general rule that maps inputs to outputs. Different models, i.e., hypothesis, can be employed in the generalization process. For the best performance in the generalization, the complexity of the hypothesis should match the complexity of the function underlying the data.

Machine learning models can be categorized as either supervised or unsupervised. Supervised learning involves learning a function that maps an input to an output based on example input-output pairs. Supervised models can be sub-categorized as either a regression or classification models. In regression models, the output is continuous. Common types of regression models include linear regression, decision trees, random forest, and neural network. In classification models, on the other hand, the output is discrete. Common types of classification models include logistic regression, support vector machine, naïve bayes, decision tree, random forest, and neural network. In the context of certain methods disclosed in the present application, the machine learning models are classification models as the output is whether or not a cancer subject is likely to respond to cetuximab.

Unsupervised learning, on the other hand, is to draw inferences and find patterns from input data without references to labeled outcomes. Two main methods used in unsupervised learning include clustering and dimensionality reduction.

Clustering is an unsupervised technique that involves the grouping, or clustering, of data points. Common clustering algorithm include k-means clustering, hierarchical clustering, mean shift clustering, and density-based clustering.

Dimensionality reduction is a process of reducing the number of random variables to obtain a set of principle variables. Common dimensionality reduction algorithms include principal component analysis (PCA), regularized regression and Boruta.

### Classification Models

As discussed above, in classification models, the output is discrete. In certain embodiments, the methods disclosed herein involve a classification model, i.e., a machine learning classifier. In certain embodiments, the machine learning classifier is built by a logistic regression model.

### Logistic Regression

The simplest idea of linear regression is to find a line that best fits the data. Extensions of linear regression include multiple linear regression (e.g., finding a plane of best fit) and polynomial regression (e.g., finding a curve of best fit). Logistic regression is similar to linear regression but is used to model the probability of a finite number of outcomes, typically two.

### Support Vector Machine

A Support Vector Machine (SVM) is a supervised classification technique that, at the most fundamental level, find a hyperplane or a boundary between two classes of data that maximizes the margin between the two classes. There are many planes that can separate the two classes, but only one plane can maximize the margin or distance between the classes.

### Decision Tree

A decision tree is a decision support tool that uses a tree-like model of decisions and their possible consequences, including chance event outcomes, resource costs, and utility. Typically, a decision tree is a flowchart-like structure in which each internal node represents a “test” on an attribute, each branch represents the outcome of the test, and each leaf node represents a class label (decision taken after computing all attribute). The paths from root to leaf represent classification rules. Decision trees are intuitive and easy to build but fall short when it comes to accuracy.

### Random Forest

Random forests are an ensemble learning technique that builds off of decision trees. Random forests involve creating multiple decision trees using bootstrapped datasets of the original data and randomly selecting a subset of variables at each step of the decision tree. The model then selects the mode of all of the predictions of each decision tree. By relying on a “majority wins” model, it reduces the risk of error from an individual tree.

### Artificial Neural Network

Artificial neural networks (ANNs), usually simply called neural networks (NNs), are inspired by the biological neural networks that constitute animal brains. An ANN is based on a collection of connected units or nodes called artificial neurons, which loosely model the neurons in a biological brain. Each connection, like the synapses in a biological brain, can transmit a signal to other neurons. An artificial neuron receives a signal then processes it and can signal neurons connected to it. The “signal” at a connection is a real number, and the output of each neuron is computed by some non-linear function of the sum of its inputs. The connections are called edges. Neurons and edges typically have a weight that adjusts as learning proceeds. The weight increases or decreases the strength of the signal at a connection. Neurons may have a threshold such that a signal is sent only if the aggregate signal crosses that threshold. Typically, neurons are aggregated into layers. Different layers may perform different transformations on their inputs. Signals travel from the first layer (the input layer), to the last layer (the output layer), possibly after traversing the layers multiple times.

### Dimensionality Reduction

Dimensionality reduction is the process of reducing the number of random variables under consideration by obtaining a set of principal variables. In the field of diagnostics data mining, the process of dimensionality reduction reduces the number of biomarkers that can be used in the diagnosis. Dimensionality reduction processes can be divided into feature selection and feature extraction. In certain embodiments, the machine learning classifier is built by dimensionality reduction using a regularized linear regression method.

### Feature Selection

Feature selection approaches, also known as variable selection, attribute selection or variable subset selection, try to find a subset of the input variables for use in model construction. The simplest algorithm of feature selection is to test each possible subset of features and find the one which minimizes the error rate. Feature selection algorithms can be divided into three categories: wrappers, filters and embedded methods.

Wrapper methods use a predictive model to score feature subsets. Each new subset is used to train a model, which is tested on a hold-out set. Counting the number of mistakes made on that hold-out set (the error rate of the model) gives the score for that subset. As wrapper methods train a new model for each subset, they are very computationally intensive, but usually provide the best performing feature set for that particular type of model or typical problem.

Filter methods use a proxy measure instead of the error rate to score a feature subset. This measure is chosen to be fast to compute, while still capturing the usefulness of the feature set. Common measures include the mutual information, the pointwise mutual information, Pearson product-moment correlation coefficient, Relief-based algorithms, and inter/intra class distance or the scores of significance tests for each class/feature combinations. Filters are usually less computationally intensive than wrappers, but they produce a feature set which is not tuned to a specific type of predictive model. This lack of tuning means a feature set from a filter is more general than the set from a wrapper, usually giving lower prediction performance than a wrapper. However, the feature set doesn't contain the assumptions of a prediction model, and so is more useful for exposing the relationships between the features. Many filters provide a feature ranking rather than an explicit best feature subset, and the cut-off point in the ranking is chosen via cross-validation. Filter methods have also been used as a preprocessing step for wrapper methods, allowing a wrapper to be used on larger problems. One other popular approach is the Recursive Feature Elimination algorithm, commonly used with Support Vector Machines to repeatedly construct a model and remove features with low weights.

Embedded methods are a catch-all group of techniques which perform feature selection as part of the model construction process. The exemplar of this approach is the regularized regression method, e.g., the LASSO (least absolute shrinkage and selection operator) method for constructing a linear model, which penalizes the regression coefficients with an L1 penalty, shrinking many of them to zero. Any features which have non-zero regression coefficients are “selected” by the LASSO algorithm. As a result, the LASSO method performs both feature selection and regularization in order to enhance the prediction accuracy and interpretability of the resulting machine learning model. Improvements to the LASSO include Bolasso which bootstraps samples; Elastic net regularization, which combines the L1 penalty of LASSO with the L2 penalty of ridge regression; and FeaLect which scores all the features based on combinatorial analysis of regression coefficients. AEFS further extends LASSO to nonlinear scenario with autoencoders. These approaches tend to be between filters and wrappers in terms of computational complexity.

### Feature Extraction

Feature extraction is a process of building from initial features a set of derived features intended to be informative and non-redundant, thus facilitating the subsequent learning and generalization steps. Examples of feature extraction algorithm include principle component analysis (PCA), isomap, partial least squares, nonlinear dimensionality reduction, multilinear subspace learning, semidefinite embedding, and autoencoder.

### Principle Component Analysis (PCA)

PCA involves project higher dimensional data to a smaller space, which results in a lower dimension of data while keeping all original variables in the model. PCA can be thought of as fitting a p-dimensional ellipsoid to the data, where each axis of the ellipsoid represents a principal component. If some axis of the ellipsoid is small, then the variance along that axis is also small.

To find the axes of the ellipsoid, the values of each variable in the dataset are first centered on 0 by subtracting the mean of the variable's observed values from each of those values. These transformed values are used instead of the original observed values for each of the variables. Then, the covariance matrix of the data is computed, and the eigenvalues and corresponding eigenvectors of this covariance matrix are calculated. Each of the orthogonal eigenvectors is then normalized to turn them into unit vectors. Once this is done, each of the mutually orthogonal unit eigenvectors can be interpreted as an axis of the ellipsoid fitted to the data. This choice of basis will transform the covariance matrix into a diagonalised form with the diagonal elements representing the variance of each axis. The proportion of the variance that each eigenvector represents can be calculated by dividing the eigenvalue corresponding to that eigenvector by the sum of all eigenvalues. In simplified terms, PCA is a method that brings together (1) a measure of how each variable (e.g., a biomarker) is associated with one another using a covariance matrix; (2) the directions in which the data are dispersed using eigenvectors; and (3) the relative importance of these different directions using eigenvalues.

### Computer-Implemented Methods, Systems and Devices

Any of the methods described herein may be totally or partially performed with a computer system including one or more processors, which can be configured to perform the steps. Thus, embodiments are directed to computer systems configured to perform the steps of any of the methods described herein, potentially with different components performing a respective step or a respective group of steps. Although presented as numbered steps, steps of methods herein can be performed at a same time or in a different order. Additionally, portions of these steps may be used with portions of other steps from other methods. Also, all or portions of a step may be optional. Any of the steps of any of the methods can be performed with modules, circuits, or other means for performing these steps.

Any of the computer systems mentioned herein may utilize any suitable number of subsystems. In some embodiments, a computer system includes a single computer apparatus, where the subsystems can be the components of the computer apparatus. In other embodiments, a computer system can include multiple computer apparatuses, each being a subsystem, with internal components. The subsystems can be interconnected via a system bus. Additional subsystems include, for examples, a printer, keyboard, storage device(s), monitor, which is coupled to display adapter, and others. Peripherals and input/output (I/O) devices, which couple to I/O controller, can be connected to the computer system by any number of means known in the art, such as serial port. For example, serial port or external interface (e.g. Ethernet, Wi-Fi, etc.) can be used to connect computer system to a wide area network such as the Internet, a mouse input device, or a scanner. The interconnection via system bus allows the central processor to communicate with each subsystem and to control the execution of instructions from system memory or the storage device(s) (e.g., a fixed disk, such as a hard drive or optical disk), as well as the exchange of information between subsystems. The system memory and/or the storage device(s) may embody a computer readable medium. Any of the data mentioned herein can be output from one component to another component and can be output to the user.

A computer system can include a plurality of the same components or subsystems, e.g., connected together by external interface or by an internal interface. In some embodiments, computer systems, subsystem, or apparatuses can communicate over a network. In such instances, one computer can be considered a client and another computer a server, where each can be part of a same computer system. A client and a server can each include multiple systems, subsystems, or components.

It should be understood that any of the embodiments of the present disclosure can be implemented in the form of control logic using hardware (e.g., an application specific integrated circuit or field programmable gate array) and/or using computer software with a generally programmable processor in a modular or integrated manner. As used herein, a processor includes a multi-core processor on a same integrated chip, or multiple processing units on a single circuit board or networked. Based on the disclosure and teachings provided herein, a person of ordinary skill in the art will know and appreciate other ways and/or methods to implement embodiments of the present disclosure using hardware and a combination of hardware and software.

Any of the software components or functions described in this application may be implemented as software code to be executed by a processor using any suitable computer language such as, for example, Java, C++ or Perl using, for example, conventional or object-oriented techniques. The software code may be stored as a series of instructions or commands on a computer readable medium for storage and/or transmission, suitable media include random access memory (RAM), a read only memory (ROM), a magnetic medium such as a hard-drive or a floppy disk, or an optical medium such as a compact disk (CD) or DVD (digital versatile disk), flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium according to an embodiment of the present invention may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

The following examples are provided to better illustrate the claimed invention and are not to be interpreted as limiting the scope of the invention. All specific compositions, materials, and methods described below, in whole or in part, fall within the scope of the present invention. These specific compositions, materials, and methods are not intended to limit the invention, but merely to illustrate specific embodiments falling within the scope of the invention. One skilled in the art may develop equivalent compositions, materials, and methods without the exercise of inventive capacity and without departing from the scope of the invention. It will be understood that many variations can be made in the procedures herein described while still remaining within the bounds of the present invention. It is the intention of the inventors that such variations are included within the scope of the invention.

### Example 1

This example shows the identification of biomarkers for predicting response to cetuximab in patient-derived xenograft (PDX) models.

### Materials and Methods

A cohort of 207 PDX models of 5 cancer types, including colon cancer, esophagus cancer, gastric cancer, head and neck cancer and lung cancer, were used in this study. Those models were subject to cetuximab treatment for at least two weeks. Both TGI and median AUC (area under curve) ratio, a newly developed metric to evaluate drug efficacy, were calculated. Those models were then divided into three categories according to median AUC ratio: 53 models responded to cetuximab treatment (AUCr<0.3 and TGI>0.7), 82 models partially responded to cetuximab treatment (0.3<AUCr<0.7 and 0.3<TGI<0.7) and 72 models did not respond to cetuximab treatment (AUC>0.7 and TGI<0.3). The response categories for each cancer type are shown in Table 2.

The genome wide gene expression level in the grafts were measured using RNA-seq. The genes with low expression levels or small variation of expression levels are removed. Then the normalized expression levels are used as the input for feature selection and modeling process.

The inventors then identified differentially expressed genes (DEGs) for responders vs. non-responders for each cancer type, defining commonly up-regulated genes as those up-regulated in at least three cancer types. The inventors found that 26 out of 52 gene functions in epidermis development. The inventors found that EGFR is the top related gene for all sample combined. Looking into different cancer types, higher EGFR expression correlates with better efficacy for ES, GA and LU but not significantly correlates with CR and HN (which may be due to limited expression range). The results indicate that EGFR expression is a good biomarker but is not enough for determining cetuximab responsiveness for some cancer types.

According to ROC (receiving operating characteristic), the expression of the following ten genes were most related to cetuximab response: FMOD, REPIN1, PTPRN2, FOXA2, C20orf56, EGFR, LY6D, SFN, MICALL1, IL1A. Other highly related genes include: TREM2, KLK5, SPRR2D, LCE2B, FAM25B, NLRP10, LCE2A, SFN, DSG3, DEFB103B, ANXA8, LCE3E, TMEM40, ANXA8L2, S10A7A.

The inventor also conducted mutation data analysis using WES data for 205 models following the method of CancerGenomeInterpreter database. The inventors found that the following gene mutations significantly correlate with cetuximab response: APC, LPP, KRAS, GNAS, TRRAP, HERC2, MACF1, CDKN2A, ABCB1, NCOR2.

Yang M et al. reported that the combination of APC and TP53 mutations can be used to predict cetuximab response (Yang M, Schell M J, Loboda A, et al. Repurposing EGFR inhibitor utility in colorectal cancer in mutant APC and TP53 subpopulations[J]. Cancer Epidemiology and Prevention Biomarkers, 2019, 28(7): 1141-1152). However, the inventors found that among 25 TP53 and APC double mutated models, only two are responders and 14 are non-responders, indicating that combination of APC and TP53 mutations is not a good predictor of cetuximab response.

The inventors also found that more than 25% of non-responders carrying KRAS mutation (see Table 3).

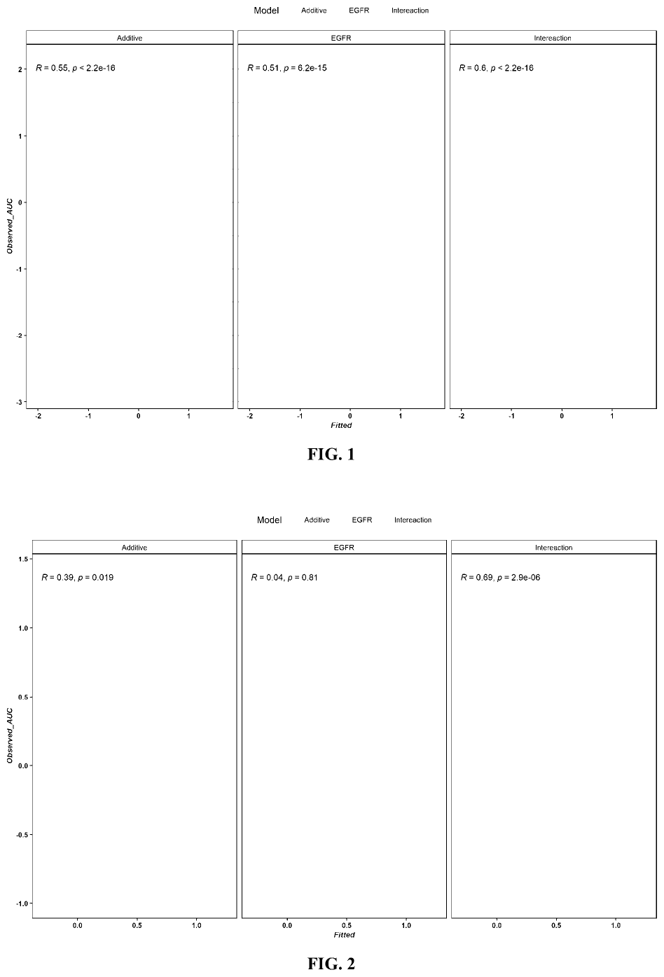

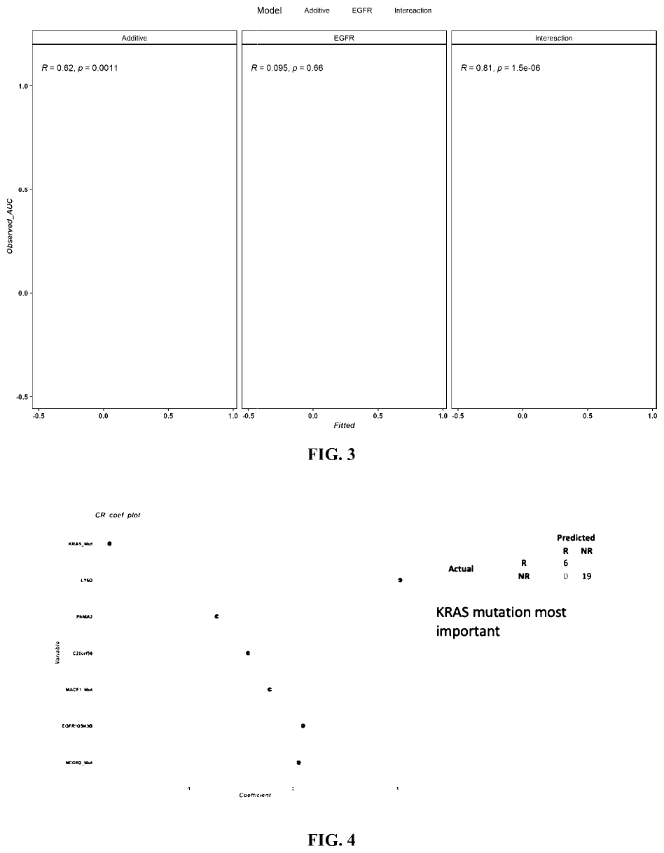

The inventors then tested the gene interaction in EGFR signaling pathway. There are 75 genes in EGFR signaling pathway that pass the low expression filter. The inventors used linear mixed model to model second order (two genes interaction) for cetuximab treatment in gastric cancer, colon cancer and neck and head cancer. The inventors calculated AIC, coefficient and p-value of two genes interaction term on treatment. The results showed that EGFR interacted with SRC, GSK3B, EIP4EBP1, AKT3 and SHC3 with both high significance and good model performance. The inventors built three linear models with genes including EGFR, SRC, GSK3B, EIF4EBP1, AKT3 and SHC3: (1) model EGFR: AUC˜EGFR; (2) additive model: AUC˜EGFR+other genes; (3) interaction model: AUC˜EGFR*(other genes). The results showed that adding interaction term between EGFR and other genes greatly improves model fit (see FIGS. 1-3).

The inventors then combined all cancer types for biomarker analysis based on the hypothesis that more samples would give higher power.

To selected candidate biomarkers, the inventors first selected the following four sets of genes/pathways: (1) high correlation genes: ranking genes by correlation (SCC between gene expression and AUCr); (2) high ROC genes: ranking genes by model performance (ROC metric based on categorical endpoints); (3) high correlation pathways: ranking pathways by correlation (SCC between GSVA score and AUCr); (4) high ROC pathways: ranking pathways by model performance (ROC metric based on categorical endpoints). The inventors selected top 10 up and down regulated genes/pathways in those four aforementioned sets, take the union (a total of 64 candidate biomarkers).

The inventors used regularized regression method (LASSO) for feature selection and build a LASSO model with 15 features.

Based on the LASSO selected features, the inventors built a logistic regression model. The inventors then further used stepwise model selection method to simplify the model. Only 6 features were left (see Table 4). This simplified model has an accuracy of 0.912 (see Table 5).

The inventors then added EGFR pathway interaction term into the model. The overall accuracy is slightly higher than model without EGFR pathway interaction term (see Table 6).

The inventors further added mutational biomarkers to the model. The final model had a 100% accuracy based on logistic regression.

The inventors used partial responder data (not used for modeling), which are more difficult to predict, as the test data (82 models). If predicted probability >0.5, then defined as Responder (R). The results showed that predicted responder(R) has lower AURr and higher TGI than NR.

The inventors also tested the model performance based on resampling method. Resample for 100 times, each time randomly select 80% samples as training dataset, and 20% samples as test dataset, calculate overall accuracy for each resample. The results were compared to EGFR+KRAS mutation model. While the average accuracy for EGFR+KRAS mutation model is about 0.8, the average accuracy for full model is about 0.9.

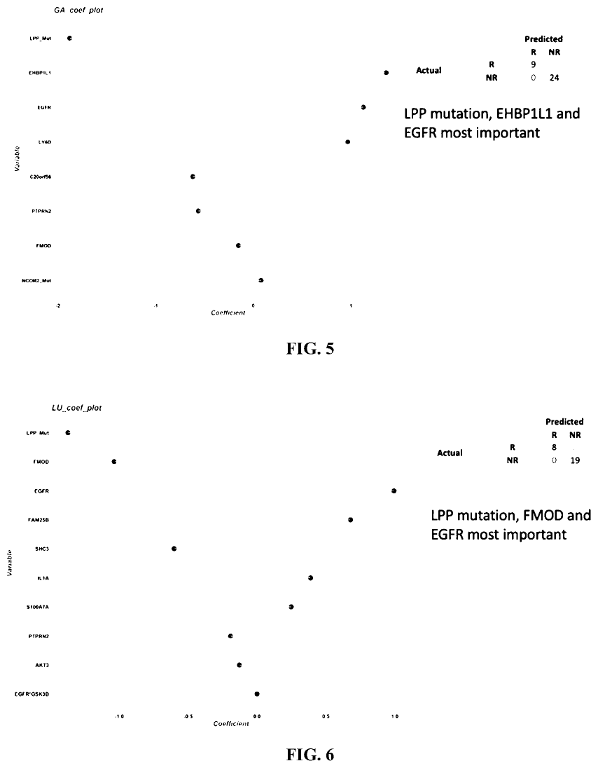

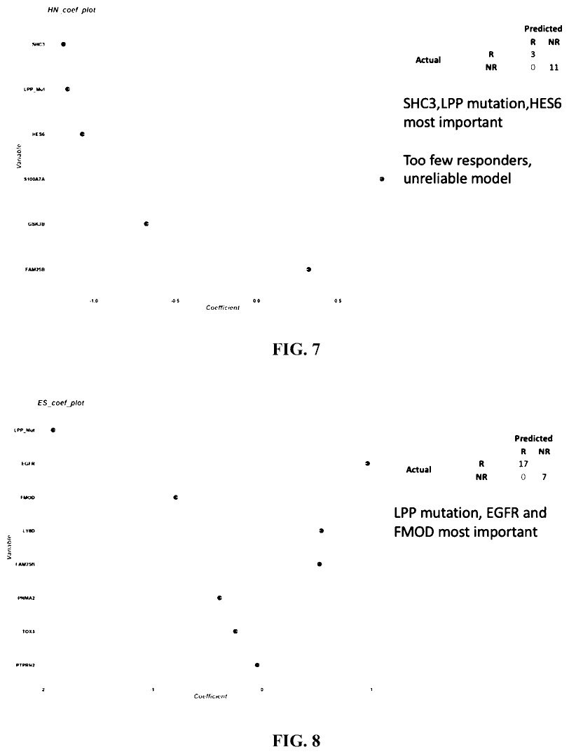

To better fit the model to each cancer type, the inventors further used samples for each cancer type to generate cancer type specific cutoff, thus generating individual model for each cancer type (see FIGS. 4-8).

TABLE 2
Response Category in Each Cancer Type

| | Response Category | | | |
|---|---|---|---|---|
| | | Non Responder | Partial Responder | Responder |
| Cancer | CR | 19 | 11 | 6 |
| Type | ES | 7 | 18 | 17 |
| | GA | 24 | 18 | 9 |
| | HN | 3 | 10 | 11 |
| | LU | 19 | 25 | 10 |

TABLE 3
Correlation of KRAS Mutations and Cetuximab Response.

| | | Cancer Type | | | | |
|---|---|---|---|---|---|---|
| | Erbitux response | CR | ES | GA | HN | LU |
| KRAS_wt | Non_Responder | 7 | 7 | 21 | 2 | 15 |
| | Partial_responder | 3 | 16 | 16 | 9 | 18 |
| | Responder | 2 | 17 | 9 | 11 | 10 |
| KRAS_mut | Non_Responder | 12 | 0 | 3 | 0 | 3 |
| | Partial_responder | 8 | 2 | 2 | 1 | 7 |
| | Responder | 4 | 0 | 0 | 0 | 0 |

TABLE 4
Features in a Simplified Model

| Feature | Coefficient | p value |
|---|---|---|
| (Intercept) | −0.54622 | 0.098047 |
| ACP6 | −0.6342 | 0.087157 |
| PNMA2 | −0.63664 | 0.097004 |
| BIOCARTA_ERBB3_PATHWAY | 0.989205 | 0.014437 |
| WP_THERMOGENESIS | −0.48397 | 0.154244 |
| GOBP_ECTODERMAL_PLACODE_FORMATION | −0.80582 | 0.043267 |
| GOBP_CD4_POSITIVE_ALPHA_BETA_T_CELL_CYTOKINE_PRODUCTION | 1.00712 | 0.01488 |

TABLE 5
Performance of a Simplified Model

Predicted

| | | R | NR |
|---|---|---|---|
| Actual | R | 46 | 4 |
| | NR | 7 | 68 |

Accuracy: 0.912
Sensitivity: 0.9200
Specificity: 0.9067
Pos Pred Value: 0.8679
Neg Pred Value: 0.9444

TABLE 6

Predicted

| | | R | NR |
|---|---|---|---|
| Actual | R | 48 | 5 |
| | NR | 5 | 67 |

Accuracy: 0.92
Sensitivity: 0.9057
Specificity: 0.9306
Pos Pred Value: 0.9057
Neg Pred Value: 0.9306

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

What is claimed is:

1. A method for predicting cetuximab sensitivity in a patient having cancer, the method comprising:
measuring in a tumor sample from the patient a set of biomarkers comprising: EGFR expression level, TMEM40 expression level, ILIA expression level, PTPRN2 expression level, LCE2A expression level, TREM2 expression level, LY6D expression level, TMEM63B expression level, EIF4EBP1 expression level, KRAS mutation, APC mutation, MACF1 mutation, NCOR2 mutation, and LPP mutation; and
determining a likelihood of the patient being responsive to cetuximab based on the measured set of biomarkers using a machine learning classifier.

2. The method of claim 1, wherein the cancer is selected from colon cancer, gastric cancer, lung cancer, head and neck cancer and esophagus cancer.

3. The method of claim 1, wherein the set of biomarkers further comprises: C20orf56 expression level, SHC expression level, DSG3 expression level, HES6 expression level, FAM25B expression level, PNMA2 expression level, GSK3B expression level, PPM1H expression level, TOX3 expression level, TYMP expression level, Anxa8L2 expression level, or ACP6 expression level.

4. A method for predicting cetuximab sensitivity in a patient having colon cancer, the method comprising:
measuring in a tumor sample from the patient a set of biomarkers comprising: KRAS mutation, LY6D expression level, PNMA2 expression level, EGFR expression level, GSK3B expression level, C20orf56 expression level, MACF1 mutation, and NCOR2 mutation; and
determining a likelihood of the patient being responsive to cetuximab based on the measured set of biomarkers using a machine learning classifier.

5. A method for predicting cetuximab sensitivity in a patient having gastric cancer, the method comprising:
measuring in a tumor sample from the patient a set of biomarkers comprising: LPP mutation, EHBP1L1 expression level, EGFR expression level, LY6D expression level, C20orf56 expression level, PTPRN2 expression level, FMOD expression level, and NCOR2 mutation; and
determining a likelihood of the patient being responsive to cetuximab based on the measured set of biomarkers using a machine learning classifier.

6. A method for predicting cetuximab sensitivity in a patient having lung cancer, the method comprising:
measuring in a tumor sample from the patient a set of biomarkers comprising: LPP mutation, FMOD expression level, EGFR expression level, GSK3B expression level, FAM25B expression level, SHC3 expression level, IL1A expression level, S10A7A expression level, PTPRN2 expression level, and AKT3 expression level; and
determining a likelihood of the patient being responsive to cetuximab based on the measured set of biomarkers using a machine learning classifier.

7. A method for predicting cetuximab sensitivity in a patient having head and neck cancer, the method comprising:
measuring in a tumor sample from the patient a set of biomarkers comprising: SHC3 expression level, LPP mutation, HES6 expression level, S100A7A expression level, GSK3B expression level, and FAM25B expression level; and
determining a likelihood of the patient being responsive to cetuximab based on the measured set of biomarkers using a machine learning classifier.

8. A method for predicting cetuximab sensitivity in a patient having esophagus cancer, the method comprising:
measuring in a tumor sample from the patient a set of biomarkers comprising: LPP mutation, EGFR expression level, FMOD expression level, LY6D expression level, FAM25B expression level, PNMA2 expression level, TOX3 expression level, and PTPRN2 expression level; and
determining a likelihood of the patient being responsive to cetuximab based on the measured set of biomarkers using a machine learning classifier.

9. The method of any one of claims 1-8, wherein the biomarkers are measured by an amplification assay, a hybridization assay, a sequencing assay or an array.

10. The method of any one of claims 1-8, wherein the machine learning classifier is built by a regularized regression method and a logistic regression model.

11. The method of any one of claims 1-8, further comprising administering cetuximab to the patient.

12. A non-transitory computer readable medium having instructions stored thereon, wherein the instructions, when executed by a processor, cause the processor to:
retrieve data of a set of biomarkers obtained from a tumor sample from a patient having a cancer, wherein the set of biomarkers comprises: EGFR expression level, TMEM40 expression level, ILIA expression level, PTPRN2 expression level, LCE2A expression level, TREM2 expression level, LY6D expression level, TMEM63B expression level, EIF4EBP1 expression level, KRAS mutation, APC mutation, MACF1 mutation, NCOR2 mutation, and LPP mutation; and
determine a likelihood of the patient being responsive to cetuximab based on the data of the set of biomarkers using a machine learning classifier.

13. The non-transitory computer readable medium of claim 12, wherein the cancer is selected from colon cancer, gastric cancer, lung cancer, head and neck cancer and esophagus cancer.

14. The non-transitory computer readable medium of claim 12, wherein the set of biomarkers further comprises: C20orf56 expression level, SHC expression level, DSG3 expression level, HES6 expression level, FAM25B expression level, PNMA2 expression level, GSK3B expression level, PPM1H expression level, TOX3 expression level, TYMP expression level, Anxa8L2 expression level, or ACP6 expression level.

15. A non-transitory computer readable medium having instructions stored thereon, wherein the instructions, when executed by a processor, cause the processor to:
retrieve data of a set of biomarkers obtained from a tumor sample from a patient having a colon cancer, wherein the set of biomarkers comprises KRAS mutation, LY6D expression level, PNMA2 expression level, EGFR expression level, GSK3B expression level, C20orf56 expression level, MACF1 mutation, and NCOR2 mutation; and
determining a likelihood of the patient being responsive to cetuximab based on the data of the set of biomarkers using a machine learning classifier.

16. A non-transitory computer readable medium having instructions stored thereon, wherein the instructions, when executed by a processor, cause the processor to:
retrieve data of a set of biomarkers obtained from a tumor sample from a patient having a gastric cancer, wherein the set of biomarkers comprises LPP mutation, EHBP1L1 expression level, EGFR expression level, LY6D expression level, C20orf56 expression level, PTPRN2 expression level, FMOD expression level, and NCOR2 mutation; and
determining a likelihood of the patient being responsive to cetuximab based on the data of the set of biomarkers using a machine learning classifier.

17. A non-transitory computer readable medium having instructions stored thereon, wherein the instructions, when executed by a processor, cause the processor to:
retrieve data of a set of biomarkers obtained from a tumor sample from a patient having a lung cancer, wherein the set of biomarkers comprises LPP mutation, FMOD expression level, EGFR expression level, GSK3B expression level, FAM25B expression level, SHC3 expression level, IL1A expression level, S10A7A expression level, PTPRN2 expression level, and AKT3 expression level; and
determining a likelihood of the patient being responsive to cetuximab based on the data of the set of biomarkers using a machine learning classifier.

18. A non-transitory computer readable medium having instructions stored thereon, wherein the instructions, when executed by a processor, cause the processor to:
retrieve data of a set of biomarkers obtained from a tumor sample from a patient having a head and neck cancer, wherein the set of biomarkers comprises SHC3 expression level, LPP mutation, HES6 expression level, S100A7A expression level, GSK3B expression level, and FAM25B expression level; and
determining a likelihood of the patient being responsive to cetuximab based on the data of the set of biomarkers using a machine learning classifier.

19. A non-transitory computer readable medium having instructions stored thereon, wherein the instructions, when executed by a processor, cause the processor to:
retrieve data of a set of biomarkers obtained from a tumor sample from a patient having a esophagus cancer, wherein the set of biomarkers comprises LPP mutation, EGFR expression level, FMOD expression level, LY6D expression level, FAM25B expression level, PNMA2 expression level, TOX3 expression level, and PTPRN2 expression level; and
determining a likelihood of the patient being responsive to cetuximab based on the data of the set of biomarkers using a machine learning classifier.

20. The non-transitory computer readable medium of any one of claims 12-19, wherein the biomarkers are measured by an amplification assay, a hybridization assay, a sequencing assay or an array.

21. The non-transitory computer readable medium of any one of claims 12-19, wherein the machine learning classifier is built by regularized regression method and a logistic regression model.

22. A method of generating a machine learning model for predicting sensitivity to an agent in a patient having a cancer, the method comprising steps of:
obtaining whole genome expression levels from each of a group of tumor models, wherein the tumor models have been tested for responsiveness to the agent;
selecting a first group of genes whose expression levels increase in the tumor models responsive to the agent when compared to the tumor models not responsive to the agent;
selecting a second group of genes whose expression levels decrease in the tumor models responsive to the agent when compared to the tumor models not responsive to the agent;
selecting a set of biomarkers from the first and the second group of genes using a regularized regression method; and
building a machine learning classifier using a logistic regression model.

23. The method of claim 22, wherein the agent is cetuximab.

24. The method of claim 22, wherein the tumor models are xenograft models.

25. The method of claim 22, wherein the first and the second group of genes are selected by correlation between gene expression level and AUCr or by model performance of ROC metric.
